(19) United States
(12) Patent Application Publication
(10) Pub. No.: US 2023/0201371 A1
(43) Pub. Date: Jun. 29, 2023

(54) COMPOSITIONS AND METHODS FOR TREATING OCULAR DISORDERS

(71) Applicant: Clearside Biomedical, Inc., Alpharetta, GA (US)

(72) Inventors: Thomas CIULLA, Alpharetta, GA (US); Cherry WAN, Alpharetta, GA (US)

(21) Appl. No.: 17/912,248

(22) PCT Filed: Mar. 18, 2021
(86) PCT No.: PCT/US2021/022980

§ 371 Date: Sep. 16, 2022

Related U.S. Application Data
(60) Provisional application No. 62/991,791, filed on Mar. 19, 2020.

(51) Int. Cl. A61K 48/00; A61K 9/00; A61K 31/56; A61K 39/395; C12N 15/113; C12N 15/86

(52) U.S. Cl. CPC A61K 48/005; A61K 9/0048; A61K 31/56; A61K 39/3955; C12N 15/113; A61K 48/0075; C12N 15/86; C12N 2310/531; C12N 2750/14143; C12N 2750/14145; C12N 2750/14171

(57) ABSTRACT

The present invention relates to compositions and methods for treating ocular disorders. More specifically, the method includes non-surgical administration of gene therapies to the suprachoroidal space of the eye of a subject who is suffering from an ocular disorder.

### CROSS-REFERENCE TO RELATED APPLICATIONS

This application claims the benefit of priority to U.S. Provisional Application No. 62/991,791, filed on Mar. 19, 2020, the entire contents of which are hereby incorporated by reference.

### FIELD OF DISCLOSURE

The present disclosure relates to compositions and methods for treating ocular disorders. More specifically, the present disclosure relates to delivery of nucleic acids to the suprachoroidal space to treat ocular disorders.

### BACKGROUND OF THE INVENTION

The eye is comprised of several specialized tissues that work together to initiate visual perception in response to photons of light. Any insult to these tissues compromises vision and impacts the quality of life for the patient. Environmental trauma, age, and genetic disorders can cause varying degrees of ocular diseases. Current therapies for ocular disorders are often surgically-based or topical treatments, however, they often fail to correct the underlying genetic deficit, and/or require repeated invasive treatments. For example, intravitreal injection of drugs is widely used to treat posterior-segment disease; however, intravitreal injection is invasive and carries the potential for infection; and the vitreous humor is not the site of action for posterior-segment disease therapies. Methods for targeting drug delivery to the chorioretinal layer, e.g., around the macula, where most posterior-segment disease is located, are desired to improve drug bioavailability and efficacy with reduced side effects.

As the eye is immune-privileged, the use of gene therapy is an attractive therapeutic option for numerous forms of ocular disorders. Accordingly, there is a need in the art for minimally invasive, targeted and effective delivery of nucleic acids as a method to treat ocular disorders.

### SUMMARY OF THE INVENTION

The disclosure relates to compositions and methods for treating an ocular disorder in a subject in need thereof. In embodiments, the disclosure relates to non-surgically administering a therapeutic agent to the suprachoroidal space (SCS) of the eye. In embodiments, the disclosure relates to non-surgically administering an effective amount of a vector encoding a transgene to the SCS of the eye.

In embodiments, the method achieves superior delivery of a therapeutic agent in the desired location of the eye and for a desired period of time. In embodiments, the method achieves exposure to the therapeutic agent at specific portions of the eye (e.g., broad expression around the entire eye, or expression targeted to the macula, depending on the indication to be treated and/or the therapeutic agent's mechanism of action); and/or a specific duration of exposure. In embodiments, the method achieves superior expression of the transgene in the desired location of the eye and for a desired period of time. In embodiments, the methods provided herein are capable of achieving gene expression at specific portions of the eye (e.g., broad expression around the entire eye, or expression targeted to the macula); and/or a specific duration of expression; and/or enhanced penetration of expression to retinal layers; and/or high gene expression with limited immune response.

Methods provided herein for achieving superior delivery of a therapeutic agent and/or superior gene expression in the eye via administration to the SCS include one or more of the following:
Expansion of the suprachoroidal space prior to SCS administration of the vector or other therapeutic agent
Manipulation of the intraocular pressure in the eye (IOP) prior to and/or following SCS administration of the vector or other therapeutic agent
Use of a pushing formulation administered prior to or concurrently with the formulation comprising the vector or other therapeutic agent; for example, use of a hydrogel pushing formulation
Immunomodulation systemically or locally (e.g., systemic or local administration of corticosteroids)
Injection of the vector or other therapeutic agent in multiple quadrants of the eye
Modulating the speed of injection of the vector or other therapeutic agent to the SCS
Employing iontophoresis to control movement of the formulation comprising the vector or other therapeutic agent within the posterior segment of the eye
Injecting the vector or other therapeutic agent in a formulation having high viscosity
Injecting the vector or other therapeutic agent in a formulation that includes hyaluronic acid or a similar agent
Injecting the vector or other therapeutic agent in a volume of about 50 μL to about 400 μL
Injecting the vector at a or other therapeutic agent high dose, for example, a high concentration of therapeutic agent or nucleic acid and/or a high concentration of viral vector comprising the nucleic acid
Injecting the vector or other therapeutic agent in a highly soluble formulation

In embodiments, the methods comprise administering a gene therapy to the SCS of the eye. In embodiments, the methods comprise administering a gene therapy to the SCS of the eye via a viral vector or non-viral particle (e.g., a DNA nanoparticle). In embodiments, the methods comprise administering a vector comprising a transgene to the SCS, wherein the transgene comprises a nucleic acid sequence which encodes a polypeptide, protein, functional RNA molecule (e.g., miRNA sponge) or other gene product of interest. In embodiments, the nucleic acid is DNA or RNA. In embodiments, the nucleic acid molecule comprises a gene sequence and a promoter sequence.

In embodiments, the nucleic acid is translated to a therapeutic agent. In further embodiments, the therapeutic agent is a protein or polypeptide. In embodiments, the nucleic acid encodes a protein selected from the group consisting of a cytokine, chemokine, a growth factor, an anti-angiogenesis factor, and an antibody or antibody fragment or construct. Antibody fragments include antigen-binding fragments selected from the group consisting of single domain antibodies, Fabs, F(ab′)2s, and single-chain variable fragments (scFv). Exemplary single domain antibodies include heavy chain-only antibodies (VHH or nanobodies). In embodiments, the antibody is a monoclonal antibody. In embodiments, the therapeutic agent is an inhibitor of VEGF, e.g., an anti-VEGF antibody, or anti-VEGF antibody fragment.

In embodiments, the nucleic acid encodes a wild-type form of a protein, where a mutant form of the protein causes an ocular disease. In embodiments, the nucleic acid encodes a protein selected from the group consisting of ABCA4, GUCY2D, RPE65, ELOVL4, CHM, CRB1, MYO7A, CDH23, CLRN1, ADGRV1, CIB2, HARS, PCDH15, USH2A, USH1C, USH1G, GPR98, WHRN, and CEP290. In embodiments, the nucleic acid encodes a therapeutic nucleotide. For example, in embodiments, the therapeutic nucleotide is shRNA or siRNA and the method is a gene knock-down approach.

In embodiments, the nucleic acid is transcribed to form transcripts and at least one of those transcripts is an anti-sense transcript which, in embodiments, is capable of inhibiting the synthesis of an endogenous protein. In other embodiments, the anti-sense transcript inhibits an endogenous protein with a dominant-negative mutation.

In embodiments, the vector used for delivering the gene is a viral vector. In embodiments, the viral vector is a vector having tropism for retinal cells and/or photoreceptor cells. In embodiments, the vector is a non-replicating recombinant adeno-associated virus vector (“AAV”). The AAV vector may comprise components from one or more AAV serotypes. In embodiments, the AAV vector is selected from AAV1, AAV2, AAV3, AAV4, AAV5, AAV6, AAV7, AAV8, AAV9, AAV10, AAV11, AAVrh10, or any combination thereof. Recombinant AAV (rAAV) vectors” of the disclosure typically comprise a transgene and its regulatory sequences, and 5′ and 3′ AAV inverted terminal repeats (ITRs). In embodiments, a transgene comprises a nucleic acid sequence which encodes a polypeptide, protein, functional RNA molecule (e.g., miRNA sponge) or other gene product of interest. The nucleic acid coding sequence is operatively linked to regulatory components in a manner that permits transgene transcription, translation, and/or expression in a cell of a target tissue.

In embodiments, other vectors may be used, including but not limited to lentiviral vectors, vaccinia viral vectors, or non-viral vectors. In embodiments, the non-viral vector is a nanoparticle.

In embodiments, the viral vector is administered at a concentration of about 10⁹ to about 10¹⁵ gene copy (GC) per eye. For example, in embodiments, the viral vector is administered at a concentration of about 10¹⁰ to about 10¹⁴ GC/eye. In embodiments, the viral vector is administered at a concentration of about 10⁹, about 10¹⁰, about 10¹¹, about 10¹², about 10¹³, about 10¹⁴, or about 10¹⁵ GC/eye. In embodiments, the method further comprises administering a steroid or other anti-inflammatory agent prior to, concurrently with, and/or subsequently to administration of the vector. In embodiments, the administration of the steroid or other anti-inflammatory agent reduces inflammation that may otherwise be associated with the high dose of the gene therapy vector. In embodiments, the steroid or other anti-inflammatory agent is administered systemically, intravitreally, periocularly, or suparchoroidally.

In embodiments, the formulation is administered to the SCS via a hollow microneedle. In embodiments, the method comprises multiple SCS administrations of the vector. In embodiments, the method comprises administration to multiple quadrants of the eye.

In embodiments, the ocular cell transfected by the gene therapy agent is a cell of the retina, retinal pigment epithelium, fovea, macula, ganglion cell layer, inner plexiform layer, inner nuclear layer, outer plexiform layer, outer nuclear layer, outer segments or inner segments of rods and cones, epithelium of the conjunctiva, iris, ciliary body, cornea, or ocular sebaceous gland epithelia.

In embodiments, the therapeutic agent is a gene therapy, oligonucleotide, polynucleotide, aptamer, small molecule, peptide, or protein (e.g., an antibody, antibody fragment, or fusion protein).

In embodiments, one or more strategies is selected to enhance the expression of the gene of interest, and/or the targeted presence of the therapeutic agent, for a particular period of time and/or in a particular tissue and/or in a particular region of a particular tissue.

### BRIEF DESCRIPTION OF THE FIGURES

FIG. 1 provides a schematic view of various procedure and formulation modifications that can be used individually or in any combination to achieve superior results in SCS administration of gene therapies or other therapeutic agents.

FIG. 2 provides data showing that increased formulation viscosity leads to increased SCS thickness, a slower collapse of the SCS, and reduced spread.

FIG. 3 provides data showing that a hyaluronic acid formulation increased the spread (% area coverage) in the SCS.

FIG. 4 provides data showing that increased volume increased the spread, but not the thickness, of the SCS

FIG. 5 provides data showing that particle size (20 nm 10 μm) does not affect spread in the SCS.

FIG. 6 provides data showing that heavy particles fall to the posterior of the eye in supine position.

FIG. 7 provides data showing that injection of collagenase increased spread in the SCS.

FIG. 8 provides data showing that sequential injections increase posterior spread.

FIG. 9 provides data showing that iontophoresis controls spread of a formulation within the SCS.

FIG. 10 provides data showing retinal transfection following suprachoroidal administration. Gene copy concentrations in the exemplary study of the bottom panel was 7×10¹¹ to 7×10¹² copy number per eye.

### DETAILED DESCRIPTION OF THE INVENTION

The present disclosure relates to methods for enhanced suprachoroidal delivery of therapeutic agents such as vectors encoding a transgene. In an aspect, the present disclosure relates to vectors (e.g., AAV vectors or non-viral vectors), nucleic acids, compositions, kits, and methods for performing gene therapy in a manner that significantly improves transgene expression in the eye. For example, the vectors, nucleic acids, compositions, kits, and methods provide superior expression of transgene products in the posterior segment of the eye. Superior expression includes higher levels of expression, superior durability of expression, targeted expression in a particular desired location in the eye, and the like. In embodiments, the disclosure provides superior methods for treating ocular diseases using the therapeutic agents, vectors, nucleic acids, compositions, kits, and methods provided herein.

### Ocular Disorders

In one aspect, the present disclosure provides methods and compositions for treating an ocular disorder or disease. In embodiments, the ocular disorder is a retinal disease. In embodiments, the ocular disorder is selected from age-related macular degeneration (AMD), neovascular age-related macular degeneration (NVAMD), retinitis pigmentosa (RP), optic neuritis, infection, uveitis, sarcoid, sickle cell disease, retinal detachment, temporal arteritis, retinal ischemia, choroidal ischemia, choroidal ischemia, ischemic optic neuropathy, arteriosclerotic retinopathy, hypertensive retinopathy, retinal artery blockage, retinal vein blockage, glaucoma, hypotension, diabetic retinopathy, diabetic macular edema (DME), macular edema occurring after retinal vein occlusion (RVO), macular edema, and choroidal neovascularization

In embodiments, the ocular disorder or disease may be caused by a genetic defect. Examples of such ocular diseases for which a gene or genes have been identified include, but are not limited to, Usher syndrome, Stargardt disease, Bardet-Biedl syndrome, Best disease, choroideremia, gyrate-atrophy, retinitis pigmentosa, macular degeneration, Leber congenital amaurosis (Leber hereditary optic neuropathy), Blue-cone monochromacy, retinoschisis, Malattia Leventinese, Oguchi disease, or Refsum disease. These may also be referred to as genetic ocular diseases. In embodiments, the ocular disorder is one which involves a mutated or absent gene in an ocular cell such as a retinal pigment epithelial cell or a photoreceptor cell.

The disclosure provides a method for treating an ocular disease in a human, or other mammalian or other animal subject. In embodiments, the method comprises administering to the subject by SCS injection an effective amount of a therapeutic agent or a vector comprising a nucleic acid encoding a therapeutic agent or an ocular cell-specific normal gene. In embodiments, the nucleic acid is operably linked to, or under the control of, a promoter sequence which directs the expression of the product of the gene in ocular cells such that the therapeutic agent can have therapeutic effect on the ocular cells in the region of expression. In embodiments, the gene is operably linked to, or under the control of, a promoter sequence which directs the expression of the product of the gene such that it replaces the lack of expression or incorrect expression of the mutated or absent gene. In embodiments, the disclosure provides methods for treating ocular diseases provided herein, e.g., nAMD or diabetic retinopathy, that are superior to previous methods, for example in that they achieve superior targeting of a therapeutic agent to a desired region of the eye and/or achieve superior duration of the effect of the therapeutic agent. In embodiments, the methods provided herein are gene therapy methods that are superior to previous gene therapy methods, for example in that they achieve superior gene expression in targeted posterior regions of the eye and/or achieve superior circumferential distribution in the eye.

### Gene Therapy Delivery Vectors

In embodiments, the vector used for delivering the gene is a viral vector. In embodiments, the viral vector is a vector having tropism for retinal cells and/or photoreceptor cells. In embodiments, the vector is a non-replicating recombinant adeno-associated virus vector (“AAV”). The AAV vector may comprise components from one or more AAV serotypes. In embodiments, the AAV vector is selected from AAV1, AAV2, AAV3, AAV4, AAV5, AAV6, AAV7, AAV8, AAV9, AAV10, AAV11, AAVrh10, or any combination thereof. Recombinant AAV (rAAV) vectors” of the disclosure typically comprise a transgene and its regulatory sequences, and 5′ and 3′ AAV inverted terminal repeats (ITRs). In embodiments, a transgene comprises a nucleic acid sequence which encodes a polypeptide, protein, functional RNA molecule (e.g., miRNA sponge) or other gene product of interest. The nucleic acid coding sequence is operatively linked to regulatory components in a manner that permits transgene transcription, translation, and/or expression in a cell of a target tissue.

In embodiments, the transgene is in a non-viral delivery agent such as a particle, which can range in size from about 20 nm to about 10 microns. In embodiments, the particle is a nanoparticle. Nanoparticle size ranges from 20 nm to 1000 nm. In embodiments, the nanoparticle is a lipid-based nanoparticle. In embodiments, the nanoparticle is a polymer-based nanoparticle. In embodiments, the nanoparticle is a peptide or protein-based nanoparticle. In embodiments, the nanoparticle is an inorganic nanoparticle. In embodiments, the nanoparticle is a carbon-based nanoparticle. In embodiments, the nanoparticle may be a polymer-based nanoparticle. The polymeric component of the nanoparticle may comprise a polymer and or a copolymer selected from the group consisting of poly-lactic acid (PLA), poly-glycolic acid (PGA), poly-lactide-co-glycolide (PLGA) (e.g. R203H), polyesters, poly (ortho ester), poly(phosphazine), poly (phosphate ester), polyethylene glycol (PEG), triblock copolymers polycaprolactones, gelatin, collagen, poly(D,L-lysine), derivatives thereof, and combinations thereof. In embodiments, the particle is sized to allow slow dissolution once the gene therapy is administered to the suprachoroidal space. In embodiments, the such slow dissolution allows for sustained PK in the posterior segment of the eye. For example, in embodiments, the particle is in the size range of about 1 micron to about 10 microns, or about 2 microns to about 5 microns. In embodiments, the gene therapy is embedded in a polymeric particle, such as PLGA.

In embodiments, the present disclosure provides compositions comprising a delivery vector provided herein comprising a transgene for use in a method for treating an ocular disease or disorder provided herein, wherein the method comprises one or more of the procedural and/or formulation modifications provided herein.

### Nucleic Acids

The terms “nucleic acid,” “oligonucleotide,” and the like are used interchangeably herein. A “nucleic acid” or “oligonucleotide” according to the present disclosure may comprise two or more naturally occurring or non-naturally occurring deoxyribonucleotides or ribonucleotides linked by a phosphodiester linkage, or by a linkage that mimics a phosphodiester linkage to a therapeutically useful degree. According to the present disclosure, an oligonucleotide will normally be considered to be double-stranded unless otherwise obvious from the context, and a nucleic acid may be single stranded or double stranded. The therapeutic oligonucleotide may be used to express a desired protein or to function as an anti-sense moiety, and examples include a gene, cDNA, RNA, siRNA, or an shRNA. In embodiments, the nucleic acid encodes a therapeutic nucleotide. For example, in embodiments, the nucleic acid encodes an shRNA and the therapy is a gene expression “knock down” approach. In embodiments, such a therapeutic nucleotide is administered subretinally or suprachoroidally.

Additionally, an oligonucleotide or nucleic acid may contain one or more modified nucleotides; such modification may be made in order to improve the nuclease resistance of the oligonucleotide, to improve the hybridization ability (i.e., raise the melting temperature or Tm) of the resulting oligonucleotide, to aid in the targeting or immobilization of the oligonucleotide or nucleic acid, or for some other purpose. The term “nucleic acid” as used herein means either DNA or RNA, or molecules which contain both ribo- and deoxyribonucleotides. The nucleic acids include genomic DNA, cDNA and oligonucleotides including sense and anti-sense nucleic acids. The nucleic acid may be double stranded, single stranded, or contain portions of both double stranded or single stranded sequence.

In addition to the therapeutic nucleic acid, the DNA within the vector may also contain DNA sequences either before or after the therapeutic sequence for promoting high level and/or tissue-specific transcription of the nucleic acid in a particular cell in the eye, may promote enhanced translation and/or stabilization of the mRNA of the therapeutic gene, and may enable episomal replication of the transgene in eye cells. The therapeutic gene may be contained within a plasmid or other suitable carrier.

The number of therapeutic genes or nucleic acids within the vector may vary from one, two, three to many, depending on the disease being treated but preferably is one and preferably includes one or more promoters.

In embodiments, the exogenous nucleic acid used in the compositions and methods herein encodes a protein to be expressed. That is, it is the protein which is used to treat the ocular disease. In an alternative embodiment, the exogenous nucleic acid is an anti-sense nucleic acid, which will inhibit or modulate the expression of a protein. In this embodiment, the exogenous nucleic acid need not be expressed. Thus, for example, ocular tumor cells may express undesirable proteins, and the methods of the present disclosure allow for the addition of anti-sense nucleic acids to regulate the expression of the undesirable proteins. Similarly, the expression of mutant forms of a protein may cause ocular disease.

In embodiments, the exogenous nucleic acid of the present disclosure may encode a regulatory protein such as a transcription or translation regulatory protein. In this embodiment, the protein itself may not directly affect the ocular disease, but instead may cause the increase or decrease in the expression of another protein which affects the ocular disease.

In embodiments, the exogenous nucleic acid encodes a single protein. In alternative embodiments, the exogenous nucleic acid encodes more than one protein. Thus, for example, several proteins which are useful to treat an ocular disorder may be desirable; alternatively, several ocular diseases may be treated at once using several exogenous nucleic acids encoding several proteins.

In embodiments, the nucleic acid encodes a protein which is expressed, preferably constitutively expressed. In embodiments, the expression of the exogenous nucleic acid is transient; that is, the exogenous protein is expressed for a limited time. In other embodiments, the expression is long-term and/or permanent. Thus, for example, transient expression systems may be used when therapeutic proteins are to be produced for a short period; for example, certain exogenous proteins are desirable after ocular surgery or wounding. Alternatively, for on-going or congenital conditions such as diabetic retinopathy, retinitis pigmentosa, or macular degeneration, long-term and/or permanent expression may be desired.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present disclosure.

Specific targetable cells within the eye include, but are not limited to, cells located in the ganglion cell layer (GCL), the inner plexiform layer inner (IPL), the inner nuclear layer (INL), the outer plexiform layer (OPL), outer nuclear layer (ONL), outer segments (OS) of rods and cones, the retinal pigmented epithelium (RPE), the inner segments (IS) of rods and cones, the epithelium of the conjunctiva, the iris, the ciliary body, the corneum, and epithelium of ocular sebaceous glands.

In embodiments, the nucleic acids include appropriate sequences that are operably linked to the nucleic acid sequences encoding the protein or RNA to promote its expression in a host cell. “Operably linked” sequences present include both expression control sequences (e.g. promoters) that are contiguous with the coding sequences for the product of interest and expression control sequences that act in trans or at a distance to control the expression of the protein or RNA.

Expression control sequences may include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein processing and/or secretion. A great number of expression control sequences, e.g., native, constitutive, inducible and/or tissue-specific, are known in the art and may be utilized to drive expression of the gene, depending upon the type of expression desired.

For eukaryotic cells, expression control sequences typically include a promoter, an enhancer, such as one derived from an immunoglobulin gene, SV40, cytomegalovirus, etc., and a polyadenylation sequence which may include splice donor and acceptor sites. The polyadenylation sequence generally is inserted following the transgene sequences and before the 3′ ITR sequence.

The regulatory sequences useful in the constructs of the present disclosure may also contain an intron, desirably located between the promoter/enhancer sequence and the gene. One possible intron sequence is also derived from SV-40, and is referred to as the SV-40 T intron sequence. Another suitable sequence includes the woodchuck hepatitis virus post-transcriptional element.

The promoter used herein may be made from among a wide number of constitutive or inducible promoters that can express the selected gene or nucleic acid in an ocular cell. In a preferred embodiment, the promoter is cell-specific. The term “cell-specific” means that the particular promoter selected for the recombinant vector can direct expression of the selected gene is a particular ocular cell type. In embodiments, the promoter is specific for expression of the gene in RPE cells. In embodiments, the promoter is specific for expression of the gene in photoreceptor cells. In embodiments, the promoter is specific for expression of the gene in the fovea. In embodiments, the promoter is specific for expression in cone cells or rod cells.

Examples of constitutive promoters which may be included in the present disclosure include, but are not limited to, the RSV LTR promoter/enhancer, the SV40 promoter, the CMV promoter, the dihydrofolate reductase promoter, the phosphoglycerol kinase (PGK) promoter and others previously mentioned or described. Examples of RPE-specific promoters include, the RPE-65 promoter, the tissue inhibitor of metalloproteinase 3 (Timp3) promoter, the tyrosinase promoter, and the promoters described in International Patent Publication No. WO 00/15822.

Examples of photoreceptor specific promoters include, but are not limited to, the rod opsin promoter, the red-green opsin promoter, the blue opsin promoter, the inter photoreceptor binding protein (IRBP) promoter and the cGMPβ phosphodiesterase promoter, and the promoters described in International Patent Publication No. WO 98/48097. Other promoters, which may be used, are described in U.S. Pat. Nos. 5,856,152 and 5,871,982.

In embodiments, the nucleic acid encodes a therapeutic protein. In embodiments, endogenous cells in the eye are transfected via the methods provided herein and become a bio-factory producing the therapeutic agent within the target tissue. In embodiments, the therapeutic agent is a protein selected from the group consisting of a cytokine, chemokine, a growth factor, an anti-angiogenesis factor, and an antibody or antibody fragment or construct. Antibody fragments include antigen-binding fragments selected from the group consisting of single domain antibodies, Fabs, F(ab′)2s, and single-chain variable fragments (scFv). Exemplary single domain antibodies include heavy chain-only antibodies (VHH or nanobodies). In embodiments, the antibody is a monoclonal antibody. In embodiments, the therapeutic agent is an inhibitor of VEGF, e.g., an anti-VEGF antibody, or anti-VEGF antibody fragment.

In embodiments, the nucleic acid is an ocular specific gene that may be associated with a genetic disease or disorder. Ocular-specific genes or nucleic acids contemplated for use in the compositions and methods of the present disclosure include, but are not limited to: RHO, PDE6A, PDE6B, CNGA1, RPE65, RLBP1, ADGRV1, ABCR, ABCA4, CIB2, CRB1, LRAT, CRX, IP1, EFEMP1, peripherin/RDS (PRPH2), ROM1, SAG, GNAT1, RHOK, ELOV4, GUCA1A, GUCY2D, CNGA3, BCP, GCP, RCP, CNTF, BDNF, HARS, HCFH, ORF15, RPGR, WHRN, LCA5, CFH, CFB, ABCR, ACBA4, C2, C3, HTRA1, T2-TrpRS, RdCVF, NXNL1, LCA9, NPHP4, RP32, RPE65, COL11A1, GNAT2, PRPF3, SEMA4A, CORD8, HMCN1, AXPC1, CFH, CRB1, RD3, USH2A, RP28, EFEMP1, ALMS1, RP33, CNGA3, MERTK, MKS1, NPHP1, BBS5, CERKL, KCNJ13, SAG, USH2B, CRV, GNAT1, ATXN7, ARL6, IQCB1, NPHP3, RHO, CLRN1, OPA1, STGD4, MCDR2, PDE6B, WFS1, NMNAT1, SPATA7, CC2D2A, PROM1, CNGA1, WFS2, MTP, BBS7, BBS12, RP29, LRAT, CYP4V2, MCDR3, VCAN, GPR98, BSMD, PDE6A, GRM6, C2, CFB, TULP1, MDDC, BBS9, RP9, PEX1, IMPDH1, OPNlSW, CORD9, RP1, TTPA, OPA6, PXMP3, CNGB3, VMD1, KCNV2, TOPORS, INVS, DFNB31, TLR4, TRIM32, RP8, JBTS1, PHYH, PEX7, ERCC6, RNANC, PCDH15, USH1H, CDH23, RGR, RBP4, PAX2, HTRA1, ARMS2, OAT, TEAD1, USH1C, EVR3, CORS2, ROM1, BEST1, BBS1, VRN1, CABP4, LRP5, MYO7A, FZD4, C1QTNF5, MFRP, CACNA2D4, COL2A1, CODA1, RDH5, BBS10, CEP290, RB1, GRK1, STGD2, ACHM1, MCDR4, NRL, RPGRIP1, LCA3, RDH12, USH1A, TTC8, FBLN5, NR2E3, BBS4, RLBP1, ABCC6, RP22, BBS2, RPGRIP1L, CNGB1, CDH3, FHASD, CACD, GUCY2D, RCD2, AIPL1, PITPNM3, PRPF8, CORD4, UNC119, CA4, USH1G, RGS9, PRCD, FSCN2, OPA4, CORD1, C3, RAX2, RGS9BP, CRX, OPA3, PRPF31, JAG1, MKKS, PANK2, USH1E, OPA5, TIMP3, RP23, RS1, RP6, DMD, A1ED, OPA2, NYX, RPGR, PRD, NPD, CAC, NA1F, RP2, PGK1, CHM, TIMM8A, RP24, COD2, RP34, OPN1LW, OPN1MW, KSS, LHON, MT-TL1, MT-ATP6, MT-TH, and MT-TS2.

### Therapeutic Agents

In embodiments, the present disclosure provide improved methods for delivery of therapeutic agents to the eye, wherein the therapeutic agent is any type of drug that may be useful in treating an ocular disorder. For example, in embodiments, the therapeutic agent may be a small molecule, an oligonucleotide, an aptamer, a peptide, a protein (e.g., an antibody, an antibody fragment, or a fusion protein), or any combination thereof. As used herein, the term “therapeutic agent” includes any type of therapeutic agent including the gene therapy vectors provided herein. Exemplary therapeutic agents include steroids, tyrosine kinase inhibitors, vascular endothelial growth factor (VEGF) inhibitors, integrin inhibitors (e.g., MK-0429), and quinones (e.g., idebenone). Exemplary integrin inhibitors include inhibitors of α1β1, α2β1 αvβ1, αvβ3, αvβ5, αvβ6, αvβ8, α4β7, α4β1, α5β1, α6β1, αEβ7, or any combination thereof. Exemplary tyrosine kinase inhibitors include dasatinib, nilotinib, bosutinib, regorafenib, sorafenib, sunitinib, cediranib, axitinib, telatinib, imatinib, brivanib, pazopanib, vatalanib, gefitinib, erlotinib, lapatinib, canertinib, lestaurtinib, pelitinib, semaxanib, masitinib or tandutinib. Exemplary antibodies or fusion proteins include aflibercept, bevacizumab, and ranbizumab.

### Methods of Treatment

Current treatments for ocular diseases often require intravitreal or subretinal injections of anti-inflammatory drugs. However, ocular disorders often affect the posterior segment of the eye (e.g., choroid and retina) and therefore, specific targeting of these tissues would be more beneficial in modulating disease progression. Gene therapy for ocular eye diseases has been attempted using intravitreal and subretinal injections, each of which are associated with insufficient effectiveness and/or safety issues. There is a need in the art for safely and effectively reaching the posterior segment of the eye with minimal invasiveness for targeted gene therapy, and/or for delivery of other therapeutic agents to specific regions of the eye. Moreover, there is a need in the art for high and durable levels of gene expression in the targeted tissues. The present disclosure addresses this and other needs.

Intravitreal injections result in drugs diffusing throughout the eye, including into the lens, iris and ciliary body at the front of the eye, which for some drugs, has been associated with safety issues, such as cataracts and elevated intraocular pressure (IOP) levels. Specifically, intravitreal administration of triamcinolone (TA) has been associated with cataracts and increases in IOP levels in 20% to 60% of patients. Without wishing to be bound by theory, it is thought that SCS injection has the potential to reduce the incidence of side effects by causing the injected formulation to remain localized in the retina and choroid without substantial diffusion to the vitreous or the front portion of the eye. Thus, SCS administration of the therapeutic agents or gene therapy vectors provided herein provide a significant improvement over intravitreal injection of therapeutic agents or gene therapy formulations. Moreover, the methods provided herein, in embodiments, include methods for further controlling the IOP which include reducing the IOP during or surrounding the time of SCS administration of the agent or vector.

Subretinal injections has become an increasingly popular method used to deliver drugs to the posterior segment of the eye. Gene therapy for the treatment of posterior ocular disorders have also used this approach, and is an effective treatment for retinitis pigmentosa and Leber congenital amaurosis. In the subretinal space, the injected material is delivered directly to the plasma membrane of the photoreceptor, RPE cells, and the subretinal blebs. Complications arising from subretinal injections include subjunctival and retinal hemorrhages, acute endophthalmitis, and intraoperative macular holes. Although both subretinal and SCS injections target the posterior segment of the eye, the needles used for SCS delivery are micron size lengths, resulting in injections that are virtually painless and blood free. This minimally invasive technique allows for recurrent treatment, providing substantial advantages over intravitreal and subretinal delivery methods. The further improved methods for SCS delivery provided herein (e.g., methods for achieving superior gene expression in targeted posterior regions of the eye and/or all the way around the globe of the eye, by incorporating formulation and/or procedural modifications provided herein) provide superior gene expression and superior treatment of ocular diseases and disorders, while maintaining the substantial advantages of SCS delivery over intravitreal and subretinal delivery methods.

In embodiments, the superior administration methods provided herein comprise prior expansion of the suprachoroidal space. For example, in embodiments, a viscous solution is administered to open up the space and/or slow down the collapse of the space, prior to injection of the therapeutic formulation. For example, in embodiments, a viscous solution (e.g., a 5% carboxymethylcellulose solution) may be injected just prior to administration of the therapeutic formulation, or about 1 hour, about 6 hours, about 12 hours, about 1 day, about 2, days, about 3, days, about 4 days, about 5 days, or more prior to administration of the formulation. In embodiments, such a viscous solution may expand the suprachoroidal space and cause it to remain expanded for at least about 1 hour, at least about 6 hours, at least about 12 hours, at least about 1 day, at least about 2 days, at least about 3 days, at least about 4 days, at least about 5 days, or longer. Alternatively, a non-viscous solution can be added to expand the suprachoroidal space. A non-viscous solution may expand the suprachoroidal space and cause it to remain expanded for at least about 1 hour, at least about 6 hours, at least about 12 hours, or at least about 1 day. In embodiments, a non-viscous or a viscous solution is injected into the suprachoroidal space immediately prior to administration of the therapeutic formulation.

In embodiments, the methods include controlling the IOP which include reducing the IOP during or surrounding the time of SCS administration of the therapeutic agent (e.g., gene therapy vector). For example, the IOP of the eye may be modulated, e.g. via anterior chamber paracentesis (ACP) or anterior chamber tap (AC tap). Modulation of IOP can be performed prior to SCS administration of the therapeutic agent, or following SCS administration of the therapeutic agent, or both prior to and following administration of the therapeutic agent. Where multiple injections of the therapeutic agent are carried out (e.g., multiquadrant injections), IOP may be modulated, e.g. via AC tap, prior to and/or following each injection. In embodiments, lowering the IOP can facilitate more readily opening the SCS acutely after therapeutic injection to result in better distribution. Further, lowering the IOP may limit egress of the therapy from the SCS after injection (e.g., through the choriocapillaris and scleral routes) to yield longer duration.

In embodiments, the superior methods of delivering a therapeutic agent such as a gene therapy to the eye provided herein involve the administration of a higher concentration of viral vector and/or a higher volume of therapeutic agent. In embodiments, the methods comprising the use of a higher concentration and/or a higher volume are performed in conjunction with methods for modulating IOP provided herein. In embodiments, the methods comprising the use of a higher concentration and/or a higher volume of gene therapy agent are performed in conjunction with administration of an anti-inflammatory agent, e.g., a steroid, in order to reduce inflammation that may otherwise be associated with the high volume and/or high concentration of vector in the eye.

In embodiments, the superior methods of delivery of a therapeutic agent or gene therapy provided herein involve the use of a pushing formulation administered prior to or concurrently with the formulation comprising the agent or vector; for example, use of a hydrogel pushing formulation. In embodiments, the superior methods provided herein involve the use of iontophoresis to control movement of the formulation comprising the agent or vector within the posterior segment of the eye. In embodiments, the methods provided herein involve a combination of hydrogel pushing and iontophoresis. Hydrogel pushing formulations and the like; and iontophoresis-related methods are provided, for example, in U.S. Patent Publication No. 2020-0061357 and International Patent Application No. PCT/US2018/030688, the entire contents of each of which are hereby incorporated by reference.

In embodiments, the superior methods of administering a therapeutic agent to the eye (e.g., a gene therapy provided herein) involve multiple injections of the therapeutic agent, e.g., vector comprising the transgene. For example, the methods, in embodiments, comprise injecting the agent or vector into more than one quadrant of the eye. For example, the methods involve injecting the agent or vector into two, three, or four quadrants of the eye. In embodiments, the injection of the agent or vector in multiple quadrants of the eye achieves drug exposure or gene expression throughout the posterior segment of the eye and/or all the way around the entire globe of the eye. In embodiments, the superior methods of administering a therapeutic agent to the eye (e.g., a gene therapy provided herein) involve modulating the speed of the injection of the agent or vector into the SCS. For example, in embodiments, the speed of injection is reduced, wherein the reduced speed improves the outcome by decreasing the pressure differential between the SCS and the sclera. For example, in embodiments, the speed of the injection into the SCS is 10 μL/second. In embodiments, the speed is reduced from 10 μL/second to decrease the pressure differential between the sclera and the SCS. In embodiments, the speed of the injection into the SCS is about 2 times to about 30 times slower than the usual speed of injection, or about 5 to about 20 times slower than the usual speed of injection. For example, in embodiments, the speed of the injection is about 5 μL/second, about 5 μL/second, about 3 μL/second, about 2 μL/second, about 1 μL/second, about 0.5 μL/second, or about 0.1 μL/second.

In embodiments, adjustments to the formulation comprising the therapeutic agent or the vector encoding the transgene are made to enhance delivery of the agent to the targeted region of the eye or to enhance gene expression in the targeted region of the eye. For example, in embodiments, the agent or vector is injected in a formulation having high viscosity. In embodiments, the formulation has a viscosity of more than about 50 cps, more than about 100 cps, more than about 150 cps, more than about 200 cps, more than about 250 cps, or more than about 300 cps at low shear rate (e.g., shear rate of 0.01 (l/s) or 0.1 (l/s). For example, in embodiments, the formulation has a viscosity of about 200 cps at a shear rate of 0.01 (l/s) or about 250 cps at a shear rate of 0.01 (l/s) or about 300 cps at a shear rate of 0.01 (l/s). In embodiments, the agent or vector is injected in a formulation that includes hyaluronic acid or a similar agent.

In embodiments, the therapeutic agent or vector is injected in a formulation having non-Newtonian properties. In embodiments, the therapeutic agent or vector is injected in a formulation that is highly soluble. In embodiments, the formulation is designed to increase the solubility of the therapeutic agent. In embodiments, a formulation that is soluble is from about 1 to about 1,000 parts solvent per 1 part solute. In embodiments, a formulation that is highly soluble is less than 1 part solvent per 1 part solute. In embodiments, the methods involve increasing the molecular weight of the composition to be administered to the SCS by conjugating the therapeutic agent or vector with a large polymer. In embodiments, administration of the therapeutic agents or gene therapy vectors provided herein in conjunction with a large polymer result in longer duration in the SCS.

As used herein, “non-surgical” ocular delivery methods comprise methods for delivery that do not require general anesthesia and/or retrobulbar anesthesia (also referred to as a retrobulbar block). Alternatively or additionally, “non-surgical” ocular delivery methods do not require a guidance mechanism or visualization technologies, which are typically required for ocular delivery via a shunt or cannula. Alternatively or additionally, “non-surgical” ocular delivery methods are conducted with the devices provided herein. Alternatively or additionally, “non-surgical” ocular delivery methods are methods of administration to the posterior segment of the eye that can be performed outside of a hospital or surgery center setting, e.g., in a doctor's office or outpatient clinic.

As used herein, “surgical” ocular drug delivery includes insertion or administration of drugs by surgical means, for example, via incision to expose and provide access to regions of the eye including the posterior region, and/or via insertion of a stent, shunt, or cannula.

The non-surgical posterior ocular disorder treatment methods described herein are used in conjunction with non-surgical posterior ocular disorder treatment methods provided, for example, in U.S. Patent Publication No. US2018-0042765, US2019-0260702, US 2019-0290485, US 2019-0240336, or US2020-0061357, or in U.S. Pat. No. 9,636,332 or U.S. Pat. No. 9,788,995, the disclosures of each of which are hereby incorporated by reference in their entireties

In embodiments, the effective amount of the nucleic acid administered to the SCS provides higher safety and/or therapeutic efficacy of the nucleic acid, compared to the therapeutic efficacy of the nucleic acid when the identical dosage is administered intravitreally, subretinally, topically, intracamerally, parenterally or orally; or compared to the therapeutic efficacy of the nucleic acid when the nucleic acid is administered in the absence of the formulation or procedural methods provided herein. For example, the methods provided herein result in unexpectedly superior expression levels, durability of expression, targeting of specific locations of the eye, and safety compared to previously known methods.

The term “suprachoroidal space,” is used interchangeably with suprachoroidal, SCS, suprachoroid and suprachoroidal, and describes the potential space in the region of the eye disposed between the sclera and choroid. This region primarily is composed of closely packed layers of long pigmented processes derived from each of the two adjacent tissues; however, a space can develop in this region as a result of fluid or other material buildup in the SCS and the adjacent tissues. The “supraciliary space,” as used herein, is encompassed by the SCS and refers to the most anterior portion of the SCS adjacent to the ciliary body, trabecular meshwork and limbus. Those skilled in the art will appreciate that the suprachoroidal space frequently is expanded by fluid buildup because of some disease state in the eye or as a result of some trauma or surgical intervention. In the present description, however, the fluid buildup is intentionally created by infusion of a drug formulation into the suprachoroid to create the SCS (which is filled with drug formulation). Not wishing to be bound by theory, it is believed that the SCS region serves as a pathway for uveoscleral outflow (i.e., a natural process of the eye moving fluid from one region of the eye to the other through) and becomes a real space in instances of choroidal detachment from the sclera.

As provided throughout, in one embodiment, the methods described herein may be carried out with a hollow or solid microneedle, for example, a rigid microneedle. In embodiments, the methods described herein may be carried out with a needle having an effective length of 2000 microns or less. For example, the methods described herein may be carried out with a needle having an effective length between about 500 microns and about 2000 microns; or between about 800 microns and about 1200 microns. For example, the methods described herein may be carried out with a needle having an effective length of about 700 microns, about 800 microns, about 900 microns, about 1000 microns, about 1100 microns, about 1200 microns, about 1300 microns, about 1400 microns, or about 1500 microns. As used herein, the terms “needle” and “microneedle” describe a device having a conduit body having a base, a shaft, and a tip end suitable for insertion into the sclera and other ocular tissue and has dimensions suitable for minimally invasive insertion and drug formulation infusion as described herein. In embodiments, the needle is a straight needle from the hub to the tip. In embodiments, the needle has a “U” shape or a “J” shape or a hook shape. Both the “length” and “effective length” of the microneedle encompass the length of the shaft of the microneedle and the bevel height of the microneedle. In embodiments, the device provided herein has a needle with a diameter of 28 gauge or smaller. Accordingly, in embodiments, the non-surgical methods provided herein comprise the administration of a drug formulation to the suprachoroidal space of the eye using a device provided herein comprising a needle having an effective length between about 500 and about 2000 microns, and/or having diameter of 28 gauge or smaller. In embodiments, the device used to carry out the methods described herein comprises one of the devices disclosed in U.S. Pat. No. 9,539,139, issued Jan. 10, 2017 or International Patent Application Publication No. WO2014/179698 (Application No. PCT/US2014/036590), filed May 2, 2014 and entitled “Apparatus and Method for Ocular Injection,” or US Patent Application Publication No. 2019-0290485, each of which is incorporated by reference herein in its entirety for all purposes. In embodiments, the microneedle used to carry out the methods described herein comprises one of the devices disclosed in International Patent Application Publication No. WO2014/036009 (Application No. PCT/US2013/056863), filed Aug. 27, 2013 and entitled “Apparatus and Method for Drug Delivery Using Microneedles,” incorporated by reference herein in its entirety for all purposes. In embodiments, the microneedle is an SCS microinjector as described herein.

In embodiments, features of the devices, formulations, and methods are provided in U.S. Pat. No. 9,636,332, U.S. Patent Application Publication No. 2018-0042765, International Patent Application Publication Nos. WO2014/074823 (Application No. PCT/US2013/069156), WO2015/195842 (Application No. PCT/US2015/036299), and/or WO2017/120601 (Application No. PCT/US2017/012757), each of which is hereby incorporated by reference in its entirety for all purposes.

In one embodiment, the device used to carry out one of the methods described herein comprises the device described in U.S. Design patent application Ser. No. 29/506,275 entitled, “Medical Injector for Ocular Injection,” filed Oct. 14, 2014, the disclosure of which is incorporated herein by reference in its entirety for all purposes. In one embodiment, the device used to carry out one of the methods described herein comprises the device described in U.S. Patent Publication No. 2015/0051581 or U.S. Patent Publication No. 2017/0095339, which are each incorporated herein by reference in their entireties for all purposes. In embodiments, such a device is an SCS microinjector as described herein.

In one embodiment, the microneedle is inserted into the eye of the human patient using a rotational/drilling technique and/or a vibrating action. In this way, the microneedle can be inserted to a desired depth by, for example, drilling the microneedles a desired number of rotations, which corresponds to a desired depth into the tissue. See, e.g., U.S. Patent Application Publication No. 2005/0137525, which is incorporated herein by reference, for a description of drilling microneedles. The rotational/drilling technique and/or a vibrating action may be applied during the insertion step, retraction step, or both.

Further details of possible manufacturing techniques are described, for example, in U.S. Patent Application Publication No. 2006/0086689, U.S. Patent Application Publication No. 2006/0084942, U.S. Patent Application Publication No. 2005/0209565, U.S. Patent Application Publication No. 2002/0082543, U.S. Pat. Nos. 6,334,856, 6,611,707, 6,743,211 and PCT/US2014/36590, filed May 2, 2014, all of which are incorporated herein by reference in their entireties for all purposes.

As used herein, the terms “about” and “approximately” generally mean plus or minus 10% of the value stated. For example, about 0.5 would include 0.45 and 0.55, about 10 would include 9 to 11, about 1000 would include 900 to 1100.

In embodiments, the dose of the gene therapy provided herein has a delivered volume of at least about 20 μL, at least about 50 uL, at least about 100 μL, at least about 200 μL, at least about 250 μL, at least about 300 μL, at least about 350 μL, at least about 400 μL, at least about 450 μL, or at least about 400 μL. In one embodiment, the amount of therapeutic formulation delivered into the suprachoroidal space from the devices described herein is from about 10 μL to about 500 μL, e.g., from about 50 μL to about 150 μL. In another embodiment, from about 50 μL to about 400 μL, e.g., from about 100 μL to about 300 μL, is non-surgically administered to the suprachoroidal space. In embodiments, high volumes of the therapeutic formulation are administered (e.g., 200 μL, 250 μL, 300 μL, 350 μL, or 400 μL). In further embodiments, the high volume is administered into 2 or more separate SCS injections with time in between. In embodiments, the high volume is administered in conjunction with anterior chamber paracentesis to control IOP.

The SCS delivery methods provided herein allow for the delivery of compositions comprising a nucleic acid over a larger tissue area and to more difficult to target tissue in a single administration as compared to previously known needle devices. For example, the method provided herein allow for delivery of the compositions comprising a nucleic acid around the entire globe of the eye. In embodiments, such superior delivery methods achieve higher gene expression in the regions of the eye affected by the disease or disorder to be treated, thereby achieving a superior treatment method. For example, in embodiments, the present disclosure provides superior methods for treating a disease such as diabetic retinopathy, by achieving gene expression of a VEGF inhibitor all the way around the globe of the eye.

The terms “subject” and “patient” are used interchangeably herein. The human subject treated with the methods and devices provided herein may be an adult or a child.

The therapeutic efficacy of the nucleic acids and formulations delivered by the methods described herein and therapeutic response of the human subject can be assayed by standard means in the art, as known to those of skill in the art. In general, the therapeutic efficacy of any particular drug can be assessed by measuring the response of the human subject after administration of the drug; a drug with a high therapeutic efficacy will show a greater amelioration and/or discontinuation of symptoms than a drug with a lower therapeutic efficacy. In non-limiting examples, the efficacy of the drug formulations provided herein can be measured, for example, by observing changes in pain intensity, changes in ocular lesions (size or number), intraocular pressure, fluid accumulation, inflammation (e.g., by measuring changes in the Hackett/McDonald ocular score), ocular hypertension, and/or visual acuity. The term “resolution” as used herein refers to a return to a baseline level. For example, in embodiments, resolution of macular thickening or resolution of macular edema and the like is defined as a macular thickness of less than 300 microns in CST. In embodiments the resolution of inflammation refers to a score of zero with respect to inflammatory measures in the eye. For example, in embodiments, resolution of inflammation in the eye refers to a score of 0 of anterior chamber flare and/or anterior chamber cells and/or vitreous haze and/or other measures of eye inflammation known in the art.

The “therapeutic formulation” delivered via the methods and devices provided herein in one embodiment, is an aqueous solution or suspension, and comprises an effective amount of the drug or therapeutic agent, for example, a suspension. In embodiments, the therapeutic formulation is a fluid drug formulation. The term “therapeutic agent” as used herein encompasses vector comprising a nucleic acid. Thus, in embodiments, the “drug formulation” is a formulation comprising a nucleic acid and a viral vector or a non-viral delivery agent, and may include one or more pharmaceutically acceptable excipient materials known in the art. The term “excipient” refers to any non-active ingredient of the formulation intended to facilitate handling, stability, dispersibility, wettability, release kinetics, and/or injection of the drug. In one embodiment, the excipient may include or consist of water or saline.

The therapeutic substance in one embodiment is formulated with one or more polymeric excipients to limit therapeutic substance migration and/or to increase viscosity of the formulation. A polymeric excipient may be selected and formulated to act as a viscous gel-like material in-situ and thereby spread into a region of the suprachoroidal space and uniformly distribute and retain the drug. The polymer excipient in one embodiment is selected and formulated to provide the appropriate viscosity, flow and dissolution properties. For example, carboxymethylcellulose is used in one embodiment to form a gel-like material in the suprachoroidal space. The viscosity of the polymer in one embodiment is enhanced by appropriate chemical modification to the polymer to increase associative properties such as the addition of hydrophobic moieties, the selection of higher molecular weight polymer or by formulation with appropriate surfactants.

The dissolution properties of the therapeutic formulation in one embodiment is adjusted by tailoring of the water solubility, molecular weight, and concentration of the polymeric excipient in the range of appropriate thixotropic properties to allow both delivery through a small gauge needle and localization in the suprachoroidal space. The polymeric excipient may be formulated to increase in viscosity or to cross-link after delivery to further limit migration or dissolution of the material and incorporated drug.

Water soluble polymers that are physiologically compatible are suitable for use as polymeric excipients in the therapeutic formulations described herein, and for delivery via the methods and devices described herein include but are not limited to synthetic polymers such as polyvinylalcohol, polyvinylpyrollidone, polyethylene glycol, polyethylene oxide, polyhydroxyethylmethacrylate, polypropylene glycol and propylene oxide, and biological polymers such as cellulose derivatives, chitin derivatives, alginate, gelatin, starch derivatives, hyaluronic acid, chondroiten sulfate, dermatin sulfate, and other glycosoaminoglycans, and mixtures or copolymers of such polymers. The polymeric excipient is selected in one embodiment to allow dissolution over time, with the rate controlled by the concentration, molecular weight, water solubility, crosslinking, enzyme lability and tissue adhesive properties of the polymer.

In one embodiment, a viscosity modifying agent is present in a therapeutic formulation delivered by one of the methods and/or devices described herein. In a further embodiment, the viscosity modifying agent is polyvinyl alcohol, polyvinyl pyrrolidone, methyl cellulose, hydroxypropyl cellulose, hydroxyethyl cellulose, hydroxymethyl cellulose or hydroxypropyl cellulose. In another embodiment, the formulation comprises a gelling agent such as poly(hydroxyethylmethacrylate), poly(N-vinylpyrrolidone), polyvinyl alcohol or an acrylic acid polymer such as Carbopol.

As described above, the nucleic acids delivered to the suprachoroidal space via the methods described herein can be administered with one or more additional drugs. The one or more additional drugs, in one embodiment, are present in the same formulation as the initial nucleic acid-containing formulation. In another embodiment, the one or more additional drugs are present in a second formulation. In even a further embodiment, the second drug formulation is delivered to the patient in need thereof via a non-surgical SCS delivery method described herein. Alternatively, the second drug formulation is delivered intravitreally, intracamerally, sub-retinally, sub-tenonally, peri-ocularly, orally, topically or parenterally to the human subject. In embodiments, the additional drug is an anti-inflammatory agent, such as a steroid.

### EXAMPLES

The present disclosure is further illustrated by reference to the following Examples. However, it should be noted that these Examples, like the embodiments described above, are illustrative and are not to be construed as restricting the scope of the disclosure in any way.

### Example 1

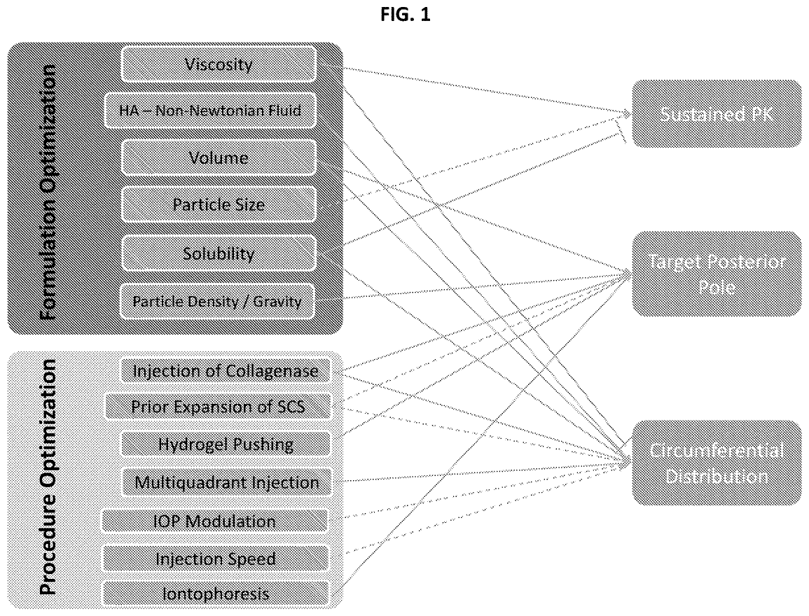

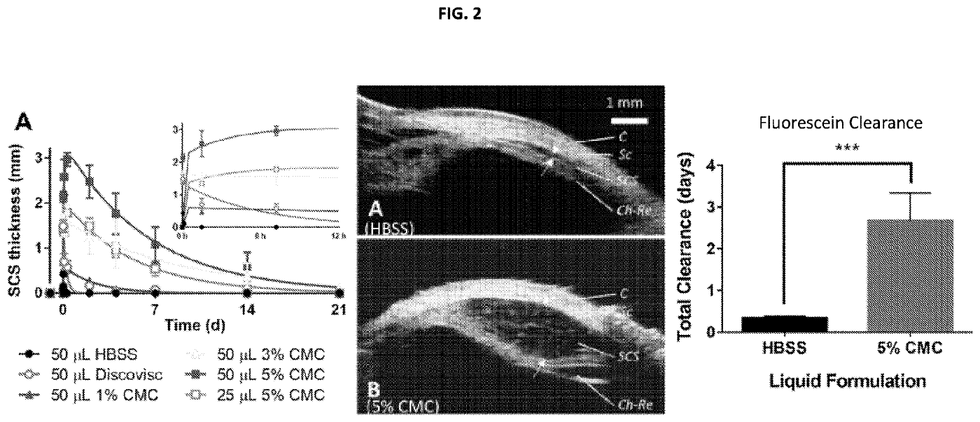

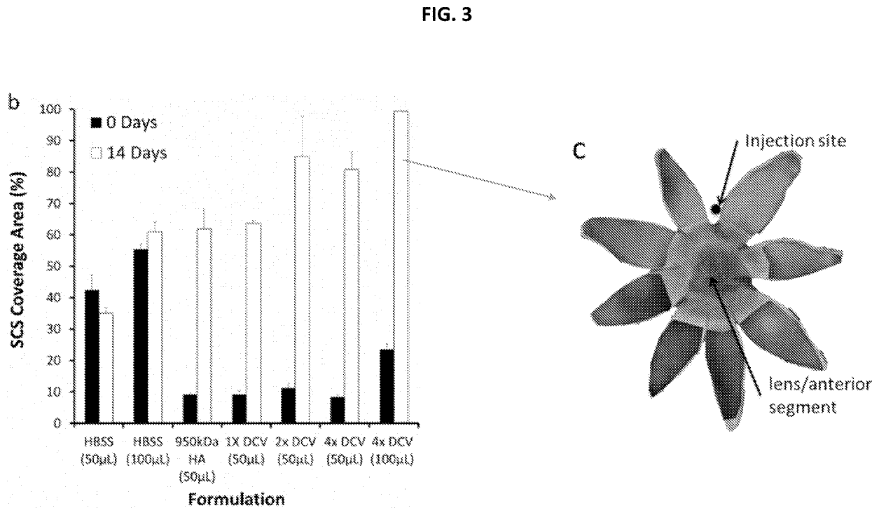

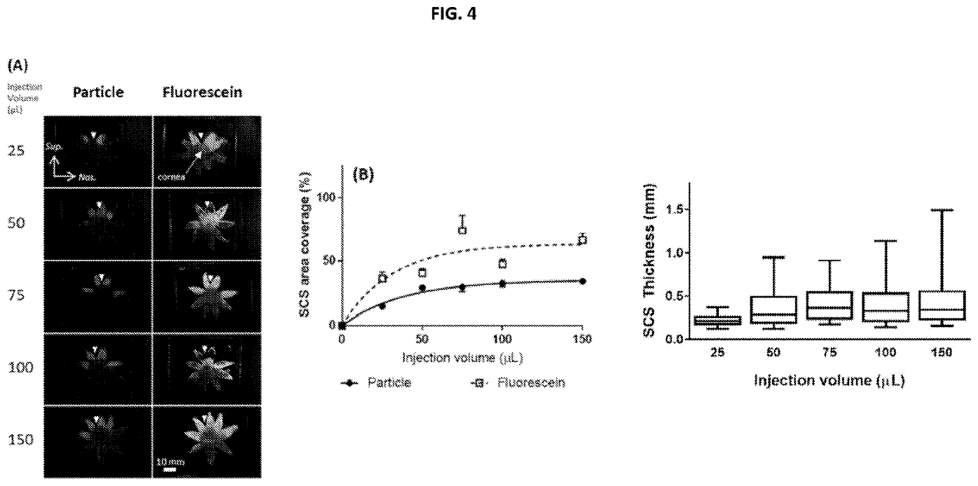

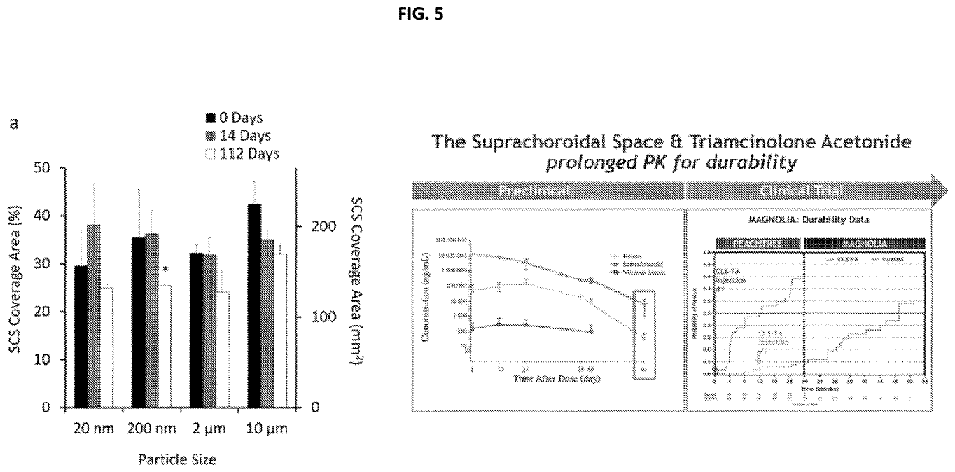

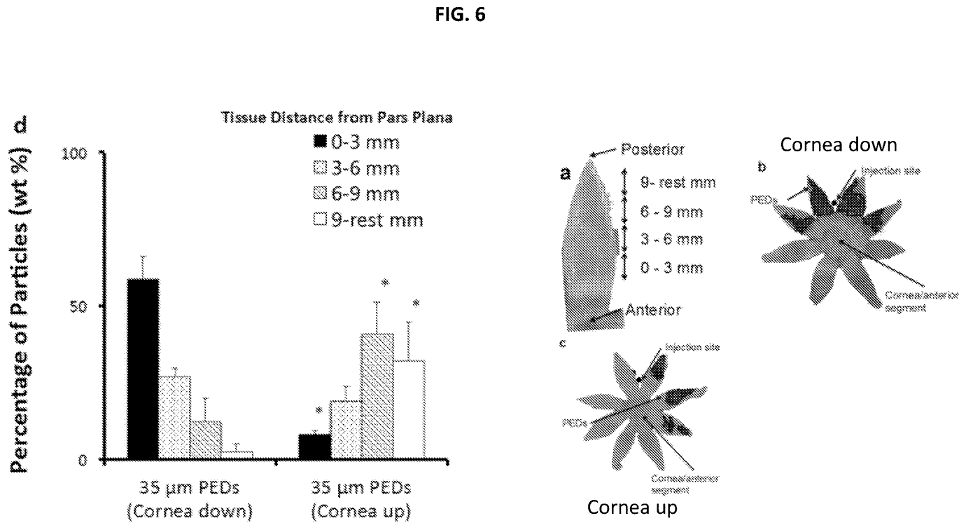

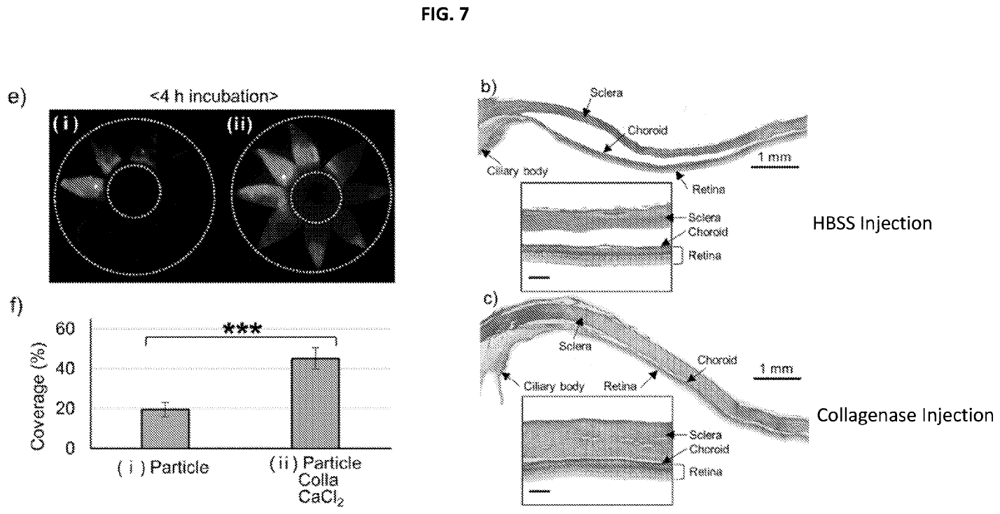

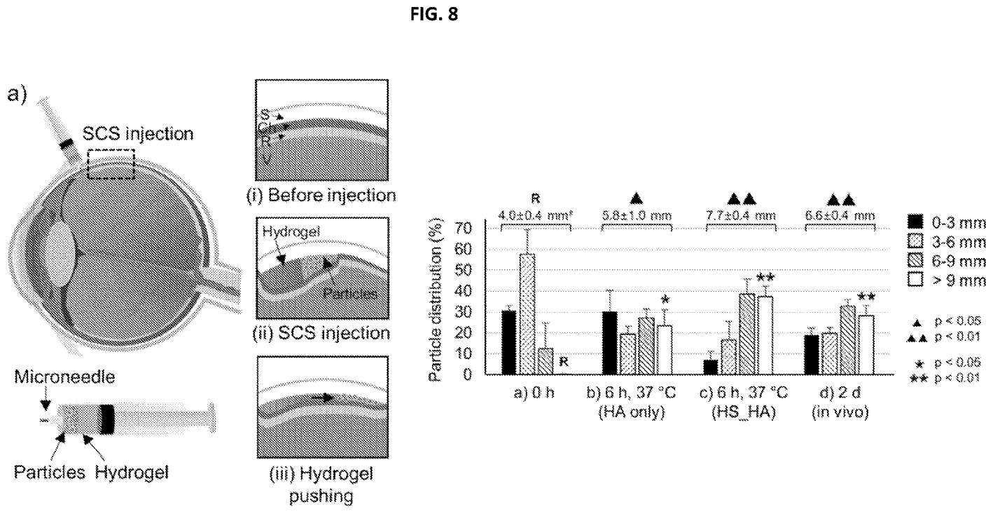

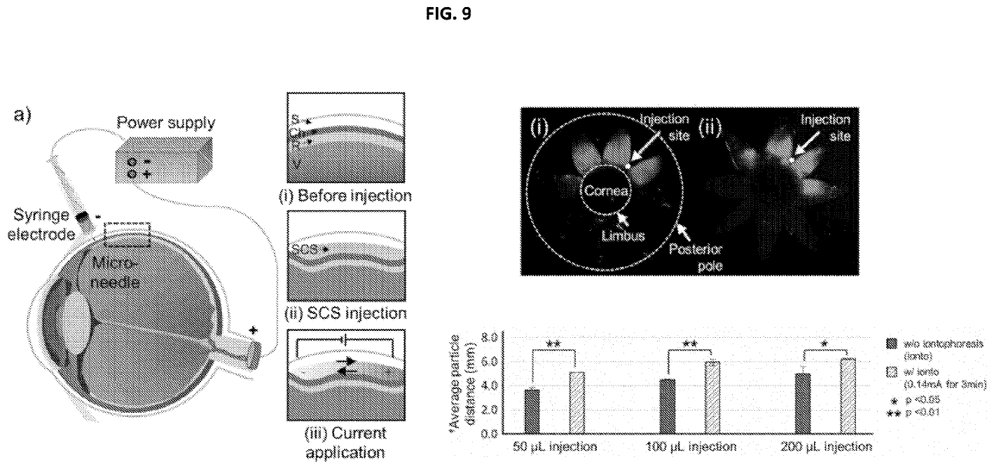

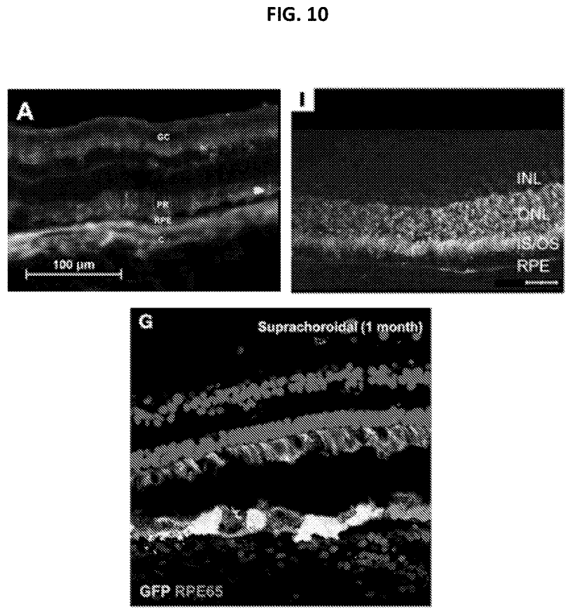

Multiple strategies are employed to provide superior methods for gene expression in the posterior segment of the eye. Exemplary strategies are provided schematically in FIG. 1. Any one or more of the formulations and/or procedures may be used to drastically improve, for example, pharmacokinetics, durability of expression, improved circumferential distribution, improved targeting of expression to the back of the eye, and/or expression around the entire globe of the eye. FIGS. 2-10 provide data showing the superior effects of some of the strategies provided herein.

Publications, patents and patent applications cited herein are specifically incorporated by reference in their entireties. While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

### REFERENCES

Chiang B, Venugopal N, Grossniklaus H E, Jung J H, Edelhauser H F, Prausnitz M R. Thickness and Closure Kinetics of the Suprachoroidal Space Following Microneedle Injection of Liquid Formulations. Invest Ophthalmol Vis Sci. 2017; 58(1):555-564. doi:10.1167/iovs.16-20377.
Kim Y C, Oh K H, Edelhauser H F, Prausnitz M R. Formulation to target delivery to the ciliary body and choroid via the suprachoroidal space of the eye using microneedles. European Journal of Pharmaceutics and Biopharmaceutics: Official Journal of Arbeitsgemeinschaft fur Pharmazeutische Verfahrenstechnik e.V. 2015 September; 95(Pt B):398-406. DOI: 10.1016/j.ejpb.2015.05.020.
Kim Y C, Edelhauser H F, Prausnitz M R. Particle-stabilized emulsion droplets for gravity-mediated targeting in the posterior segment of the eye. Adv Healthc Mater. 2014; 3(8):1272-1282. doi:10.1002/adhm.201300696.
Chiang B, Venugopal N, Edelhauser H F, Prausnitz M R. Distribution of particles, small molecules and polymeric formulation excipients in the suprachoroidal space after microneedle injection. Exp Eye Res. 2016; 153:101-109.
Chiang B, Venugopal N, Grossniklaus H E, Jung J H, Edelhauser H F, Prausnitz M R. Thickness and Closure Kinetics of the Suprachoroidal Space Following Microneedle Injection of Liquid Formulations. Invest Ophthalmol Vis Sci. 2017; 58(1):555-564. doi:10.1167/iovs.16-20377 doi:10.1016/j.exer.2016.10.011.
Gilger, et al, Treatment of Acute Posterior Uveitis in a Porcine Model by Injection of Triamcinolone Acetonide into the Suprachoroidal Space Using Microneedles, Physiology and Pharmacology.
Kim Y C, Oh K H, Edelhauser H F, Prausnitz M R. Formulation to target delivery to the ciliary body and choroid via the suprachoroidal space of the eye using microneedles. European Journal of Pharmaceutics and Biopharmaceutics: Official Journal of Arbeitsgemeinschaft fur Pharmazeutische Verfahrenstechnik e.V. 2015 September; 95(Pt B):398-406. DOI: 10.1016/j.ejpb.2015.05.020.
Jung J H, Desit P, Prausnitz M R. Targeted Drug Delivery in the Suprachoroidal Space by Swollen Hydrogel Pushing. Invest Ophthalmol Vis Sci. 2018; 59(5):2069-2079. doi:10.1167/iovs.17-23758.
Bryce Chiang, Ke Wang, C. Ross Ethier, Mark R. Prausnitz; Clearance Kinetics and Clearance Routes of Molecules From the Suprachoroidal Space After Microneedle Injection. Invest. Ophthalmol. Vis. Sci. 2017; 58(1):545-554.
Ding K, Shen J, Hafiz Z, et al. AAV8-vectored suprachoroidal gene transfer produces widespread ocular transgene expression. J Clin Invest. 2019; 130(11):4901-4911. Published 2019 Aug. 13. doi:10.1172/JCI129085.
Peden M C, Min J, Meyers C, Lukowski Z, Li Q, et al. (2011) Ab-Externo AAV-Mediated Gene Delivery to the Suprachoroidal Space Using a 250 Micron Flexible Microcatheter. PLoS ONE 6(2): e17140. doi:10.1371/journal.pone.0017140.
Yiu G, Chung S H, Mollhoff I N, et al. Suprachoroidal and Subretinal Injections of AAV Using Transscleral Microneedles for Retinal Gene Delivery in Nonhuman Primates. Mol Ther Methods Clin Dev. 2020; 16:179-191. doi:10.1016/j.omtm.2020.01.002.
Pitkdnen L, Ranta V P, Moilanen H, Urtti A. Permeability of retinal pigment epithelium: Effects of permeant molecular weight and lipophilicity. Investig Ophthalmol Vis Sci. 2005; 46(2):641-646. doi:10.1167/iovs.04-1051.
Cideciyan A V et al. Mutation-independent rhodopsin gene therapy by knockdown and replacement with a single AAV vector. PNAS 115(36) E8547-E8556 (2018).

1. A method of treating an ocular disorder in a subject in need thereof, the method comprising non-surgically administering an effective amount of a vector comprising a transgene to the suprachoroidal space (SCS) of the eye of the subject, wherein the method achieves expression of the transgene in the posterior segment of the eye.

2. The method of claim 1, wherein the vector is a viral vector, and wherein the method comprises administering the vector at a dose of about 10¹⁰ to about 10¹⁵ gene copy number per eye.

3. The method of claim 2, further comprising administering a steroid to the eye.

4. The method of claim 1, wherein the method comprises administering the vector to the suprachoroidal space of the eye in a volume of about 50 μL to about 400 μL per injection.

5. The method of claim 1, comprising administering the vector in at least two injections, wherein the injections are administered to at least two different quadrants of the eye.

6. The method of claim 5, comprising administering the vector via injection to at least three different quadrants of the eye.

7. The method of claim 6, comprising administering the vector to four different quadrants of the eye.

8. The method of claim 1, further comprising administering an anti-inflammatory agent to the subject.

9. The method of claim 8, wherein the anti-inflammatory agent is administered systemically, intravitreally, periocularly, or suprachoroidally.

10. The method of claim 8, wherein the anti-inflammatory agent is a steroid.

11. The method of claim 1, further comprising modulating the intraocular pressure of the eye prior to and/or following administration of the vector.

12. The method of claim 1, wherein the ocular disorder is diabetic retinopathy or neovascular age-related macular degeneration (nAMD), and wherein the method achieves expression of the gene around the full globe of the eye.

13. The method of claim 1, wherein the subject is a human.

14. The method of claim 1, wherein the vector is an AAV.

15. The method of claim 14, wherein the AAV is AAV8 or a variant thereof.

16. The method of claim 1, wherein the vector is a non-viral nanoparticle.

17. The method of claim 16, wherein the nanoparticle is a lipid-based nanoparticle, a polymer-based nanoparticle, a peptide, or a protein-based nanoparticle.

18. The method of claim 1, wherein the transgene encodes a therapeutic agent.

19. The method of claim 1, wherein the transgene encodes a protein selected from the group consisting of a cytokine, chemokine, a growth factor, an anti-angiogenesis factor, and an antibody or antibody fragment or construct.

20. The method of claim 1, wherein the transgene encodes a vascular endothelial growth factor (VEGF) inhibitor.

21. The method of claim 20, wherein the VEGF inhibitor is an anti-VEGF antibody or a fragment thereof.

22. The method of claim 1, wherein the transgene encodes a wild-type form of a protein, where a mutant form of the protein causes an ocular disease.

23. The method of claim 1, wherein the transgene encodes a therapeutic nucleotide.

24. The method of claim 23, wherein the therapeutic nucleotide is shRNA.

25. The method of claim 1, wherein the vector is administered to the SCS via a hollow microneedle.

26. The method of claim 1, wherein upon administration, the vector transfect an ocular cell, wherein the ocular cell is a cell of the retina, retinal pigment epithelium, fovea, macula, ganglion cell layer, inner plexiform layer, inner nuclear layer, outer plexiform layer, outer nuclear layer, outer segments or inner segments of rods and cones, epithelium of the conjunctiva, iris, ciliary body, cornea, or ocular sebaceous gland epithelia.

27. A method of treating an ocular disorder in a subject in need thereof, the method comprising non-surgically administering an effective amount of a therapeutic agent to the suprachoroidal space (SCS) of the eye of the subject, wherein the method comprises one or more of:
(i) expanding the suprachoroidal space prior to SCS administration of the therapeutic agent;
(ii) manipulating the intraocular pressure in the eye (IOP) prior to and/or following SCS administration of the therapeutic agent;
(iii) administering a pushing formulation prior to or concurrently with the therapeutic agent;
(iv) administering a steroid systemically or locally prior to or concurrently with the therapeutic agent;
(v) administering the therapeutic agent by injection in two or more quadrants of the eye;
(vi) modulating the speed of injection of the therapeutic agent into the eye;
(vii) applying iontophoresis to control movement of the therapeutic agent within the posterior segment of the eye;
(viii) injecting the therapeutic agent in a formulation having high viscosity;
(ix) injecting the therapeutic agent in a formulation that comprises hyaluronic acid;
(x) conjugating the therapeutic agent to a large polymer prior to administration;
(xi) injecting the therapeutic agent in a volume of about 50 μL to about 400 μL;
(xii) injecting the therapeutic agent at a high dose or high concentration; and
(xiii) injecting the therapeutic agent in a highly soluble formulation.
